(19) United States
(12) Patent Application Publication
(10) Pub. No.: US 2024/0252760 A1
(43) Pub. Date: Aug. 1, 2024

(54) CODED HOUSING COMPONENTS FOR AN INJECTION DEVICE

(71) Applicant: Sanofi, PARIS (FR)

(72) Inventors: Beate Franke, Frankfurt am Main (DE); Christian Frescha, Rüsselsheim (DE); Michael Helmer, Frankfurt am Main (DE); Stephan Muecke, Rüsselsheim (DE); Peter Nober, Rüsselsheim (DE); Matthias Rau, Rüsselsheim (DE); Michael Schabbach, Frankfurt am Main (DE); Martin Vitt, Frankfurt am Main (DE)

(21) Appl. No.: 18/289,267

(22) PCT Filed: May 2, 2022
(86) PCT No.: PCT/EP2022/061644

§ 371 Date: Nov. 2, 2023

(30) Foreign Application Priority Data
May 3, 2021 (EP) 21315074.1

(51) Int. Cl. A61M 5/315; A61M 5/00; A61M 5/32

(52) U.S. Cl. CPC A61M 5/3155; A61M 5/002; A61M 5/3202

(57) ABSTRACT

A coded housing of a drug delivery device includes a first housing component having a first connecting end, a second housing component having a second connecting end, an insert on one of the first connecting end or the second connecting end, a receptacle on the other one of the first connecting end or the second connecting end, a fastening element on the insert, a counter fastening element in the receptacle, a mechanical coding on the insert and including a coding feature, and a mechanical counter coding in the receptacle and including a counter coding feature. The mechanical coding is defined by a combination of a position of the coding feature with regard to a longitudinal direction and an extent of the coding feature in one or both of the longitudinal direction or a transverse direction.

### CROSS REFERENCE TO RELATED APPLICATIONS

The present application is the national stage entry of International Patent Application No. PCT/EP2022/061644, filed on May 2, 2022, and claims priority to Application No. 21315074.1, filed on May 3, 2021, the disclosures of which are incorporated herein by reference.

### TECHNICAL FIELD

The present disclosure relates to the field of drug delivery devices and systems, particularly to injection devices for injecting a liquid medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patient suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge.

Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

### SUMMARY

The present disclosure is directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

There is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device. There is a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

In one aspect, the disclosure relates to a housing of a drug delivery device, in particular to a housing of an injection device, such as a handheld injection pen. The housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. With some examples the first housing component is an elongated or tubular shaped housing component comprising the first connecting end at a longitudinal proximal end. The second housing component can be also of tubular or elongated shape. The second connecting end can be located at a distal longitudinal end of the second housing component.

There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing further comprises a fastening element provided on the insert and a counter fastening element complementary shaped to the fastening element and provided in the receptacle. Typically, and when reaching a final assembly configuration the fastening element engages the counter fastening element thereby fastening and fixing the first housing component to the second housing component; and vice versa.

The housing further comprises a mechanical coding provided on the insert. The mechanical coding comprises a coding feature. The housing further comprises a mechanical counter coding provided in the receptacle and comprising a counter coding feature. The mechanical coding and the mechanical counter coding are operable to prevent an engagement of the fastening element with the counter fastening element when the mechanical coding does not match the mechanical counter coding. In some embodiments, a mutual assembly of the first housing component and the second housing component and/or a mutual engagement of the fastening element and the counter fastening element requires that a first housing component provided with a mechanical coding is assembled with a second housing component provided with a corresponding or complementary-shaped mechanical counter coding.

In some implementations, only when the mechanical coding of the insert matches the mechanical counter coding in the receptacle the fastening element and the counter fastening element will be enabled to mutually engage. With all other pairings or combinations of a mechanical coding, e.g. of a first type, with a non-matching mechanical counter coding, e.g. of a second type, the fastening element and the counter fastening element are hindered from mutually engaging. Then, a mutual assembly and/or a fixing of first and second housing components is effectively prevented.

In some embodiments, mutual engagement of the fastening element with the counter fastening element requires that the mechanical coding matches the mechanical counter coding. In this way, unintended cross use of a first housing component of a first drug delivery device with a second housing component of another drug delivery device can be effectively prevented.

Prevention of the mutual engagement of the fastening element with the counter fastening element can be effectively achieved in two different ways. According to some examples the mechanical coding and the non-matching mechanical counter coding are configured to prevent a complete insertion of the insert into the receptacle along the longitudinal direction. Here, and before the insert reaches a final assembly position inside the receptacle the mechanical coding mechanically engages with the non-matching mechanical counter coding thereby impeding any further longitudinal movement of the insert into the receptacle. Here, the fastening element is hindered to reach an intended final assembly position in which it could engage with the complementary-shaped counter fastening element.

With other examples the fastening element is integrated into the mechanical coding and the counter fastening element is integrated into the mechanical counter coding. Here the mechanical coding non-matching with the mechanical counter coding can allow and support that the fastening element reaches a predefined final assembly position inside the receptacle but the fastening element can be hindered to engage with the counter fastening element. With this example, the fastening element may not match the respective counter fastening element. Accordingly, the fastening element is hindered to engage the counter fastening element and a mutual fastening of the first housing component and the second housing component can be effectively impeded.

Moreover, the mechanical coding is defined by a combination of a position of the coding feature with regard to the longitudinal direction and an extent of the coding feature in at least one of the longitudinal direction or a transverse direction. The longitudinal direction can be also denoted as the axial direction and the transverse direction can extend substantially perpendicular to the longitudinal direction. The transverse direction can be also regarded as a circumferential and/or as a radial direction, e.g. with regards to a tubular shape of one of the first and second housing components.

The mechanical coding is a unique combination of a position of the coding feature with regard to the longitudinal direction and extent or dimension of the coding feature in the longitudinal direction, the transverse direction, or both the longitudinal direction and the transverse direction. With some examples the mechanical coding and the mechanical coding feature is defined by a relation between the longitudinal position of the coding feature versus the longitudinal extent of the coding feature on the insert.

With other examples the mechanical coding or coding feature is defined by a relation between the longitudinal position versus the extent of the coding feature along the transverse direction on the insert. With further example the mechanical coding and the mechanical coding feature is defined by a relation between the longitudinal position versus the longitudinal extent in the transverse or circumferential extent of the respective coding feature.

Insofar, the mechanical coding and the mechanical coding feature is unequivocally defined and is characterized by at least two geometric parameters. One geometric parameter is the longitudinal position of the coding feature on the insert. The second parameter is at least one of the longitudinal extent of the coding feature on the insert or a transverse extent of the coding feature on the insert.

A coding feature of a first type distinguishes from a coding feature of a second type by the longitudinal position of the coding feature on the insert and by at least one of the longitudinal extent or the transverse extent of the coding feature on the insert.

The same requirements are also valid for the mechanical counter coding. Hence, the mechanical counter coding is also defined by a combination of a position of the counter coding feature with regard to the longitudinal direction in the receptacle and an extent of the counter coding feature in at least one of the longitudinal direction or a transverse direction or circumferential direction in the receptacle of the second housing component.

Varying of the longitudinal position of a coding feature of a first type and simultaneously varying at least one of the longitudinal or transverse extent of the respective coding feature provides a coding feature of a second type that distinguishes from coding feature of the first type. This has the benefit that a unique coding feature of the first type can only engage with a complementary-shaped counter coding feature of the first type. A coding feature of a second type is incompatible with a counter coding feature of a second type; and vice versa.

A coding feature of a second type non-matching or incompatible with the coding feature of the first type comprises an extent in the longitudinal direction or transverse direction that is larger or smaller than a respective extent of a counter coding feature of the first type. Alternatively, the longitudinal position of the coding feature of the second type is longitudinally offset from a respective counter coding feature of the first type such that a mutual engagement or matching of the coding feature of the second type with a counter coding feature of the first type is effectively impeded and/or avoided.

The twofold variation of the mechanical coding feature, i.e. with regard to the longitudinal position and with regard to at least one of the longitudinal extent or the transverse extent is beneficial to provide numerous mechanical codings and complementary shaped mechanical counter codings of different types, wherein a particular mechanical coding among numerous types of codings can only and exclusively engage with a mechanical counter coding of the same type.

The mechanical coding of the selected type is unable to engage with any other mechanical coding of any of the other types of mechanical coding.

According to another example the coding feature and the counter coding feature comprise a radial protrusion on one of the insert and the receptacle and a radial recess on the other one of the insert and the receptacle. Typically, the radial protrusion is provided on the outside surface of the insert or on an inside surface of a sidewall of the receptacle. The radial recess is provided on the other one of the outside surface of the sidewall of the insert and the inside surface of the receptacle. Upon reaching a final assembly configuration, in which the insert of the first housing component is entirely received in the receptacle of the second housing component, the radial protrusion engages with the radial recess. This engagement can be accompanied by a haptic or acoustic feedback, thus giving a user of the housing or of drug delivery device a direct feedback that the coding feature and the counter coding feature mutually engaged.

The radial extent of the radial protrusion typically matches with a radial extent of the radial recess. With a matching pair of coding features and counter coding features the radial protrusion is typically entirely received in the radial recess.

With some examples the radial protrusion is provided on the outside surface of the insert and the radial recess is provided on the inside surface of a sidewall of the insert. With other examples the radial protrusion is provided on the inside surface of the sidewall of the receptacle. Here, the radial protrusion protrudes radially inwardly. The correspondingly shaped radial recess is then provided on an outside surface of the insert of the first housing component and protrudes radially outwardly.

A radial protrusion and a radial recess can be easily manufactured and integrated on the outside surface of the insert as well as on the inside surface of the receptacle, especially when the first housing component and/or the second housing component are manufactured as an injection molded plastic component. Typically, the radial protrusion and/or the radial recess can be integrally formed in a sidewall of the first housing component and the second housing component, respectively.

According to a further example and when the mechanical coding matches the mechanical counter coding the radial protrusion fits into the radial recess. Typically, the longitudinal and/or circumferential or transverse size of a radial protrusion of one of the coding feature and counter coding feature precisely matches the inner dimensions of the radial recess. Hence, for a matching pair of a coding feature and a counter coding feature the dimensions of the radial protrusion as seen in longitudinal direction and as seen along a transverse or circumferential direction precisely match with the respective dimensions of the radial recess with regards to the longitudinal direction and with regards to the transverse or circumferential direction.

In certain implementations, only mutually matching pairs of a coding feature and a counter coding feature can engage, whereas a non-matching pair, e.g. a coding feature of a first type and a counter coding feature of a second type cannot engage.

According to another example and when the mechanical coding does not match the mechanical counter coding the radial protrusion does not fit into the radial recess. This can be due to a varying geometry of the radial protrusion and the radial recess. For instance, the extent of the radial protrusion along the longitudinal direction and/or along the transverse direction can be larger than the respective dimension of the radial recess. In such a situation the radial protrusion cannot enter the radial recess even if the radial protrusion spatially overlaps with the radial recess.

With other configurations it is conceivable that the radial protrusion comprises an extent or dimension along the longitudinal direction and/or along the transverse direction that are smaller than or equal the respective extent of the radial recess. But here and with a non-matching or incompatible pair of a coding feature of a first type with a counter coding feature of a second type the radial protrusion may not reach a position in which it at least partially overlaps or engages the radial recesses.

Here, the longitudinal position of the radial protrusion on one of the insert and the receptacle may not match with the longitudinal position of the radial recess on the other one of the insert and the receptacle. Hence, upon reaching a final assembly configuration the radial protrusion and the radial recess of the coding feature and the respective counter coding feature cannot engage and the coding features are thus non-matching.

According to a further example the coding feature is a longitudinal position versus a longitudinal extent of one of the radial protrusion and the radial recess on an outside surface of the insert. With other examples, wherein the coding feature is a longitudinal position versus a longitudinal extent of the radial protrusion on the outside surface of the insert the counter coding feature is the longitudinal position versus a longitudinal extent of the radial recess on or in the receptacle, in particular on an inside surface of a sidewall of the receptacle.

The relationship between the longitudinal position versus a longitudinal extent provides a unique identifier for the coding feature and the correspondingly or complementary-shaped counter coding feature. Moreover, a coding feature of a first type is effectively impeded and hindered to engage or to cooperate with a counter coding feature of a second type.

According to another example the coding feature is a longitudinal position versus a transverse extent of one of the radial protrusion and the radial recess on an outside surface of the insert. With further examples, wherein the coding feature is a longitudinal position versus a transverse extent of the radial protrusion on an outside surface of the insert the complementary shaped counter coding feature is a longitudinal position versus a transverse extent of the radial recess in the receptacle, typically a radial recess in a sidewall of the receptacle. Also here, the relationship between the longitudinal position versus the transverse extent provides a unique identification of the coding feature and of the respective counter coding feature. A coding feature of a first type is thereby hindered to engage or to cooperate with a counter coding feature of a second type.

According to a further example the coding feature and the counter coding feature distinguish from coding features and counter coding features of another housing, e.g. of a housing of a second type, by varying the longitudinal position of the radial recess and the longitudinal position of the radial protrusion correspondingly. Here, an increase of a longitudinal distance of the radial recess from a free end of the first or second connecting end is accompanied by or combined with an increase of the longitudinal extent and/or of the transverse extent of the radial recess and by a corresponding increase of the longitudinal extent and/or the transverse extent of the corresponding radial protrusion.

Here, an increase of a longitudinal distance of the radial recess from a free end of one of the first and second connecting end is also combined or accompanied by a corresponding decrease of a distance of the radial protrusion from the other one of the first and second connecting end and by a corresponding increase of the longitudinal extent and/or transverse extent of the radial protrusion. In this way, the modified longitudinal position of the radial protrusion is adapted to the modified longitudinal position of the radial recess.

When starting for instance from a coding feature and a complementary-shaped counter coding feature of a first type a second type of a coding feature and a counter coding feature can be obtained by increasing of the longitudinal distance of the radial recess from the free end of one of the first or second connecting end and by a corresponding increase of the longitudinal extent and/or transverse extent of the corresponding radial protrusion. In this way it can be guaranteed that the radial protrusion of the first type is hindered to engage with the radial recess of the second type. Vice versa, the radial protrusion of the second type is hindered to engage with the radial recess of the first type. In some instances, with this type of a mechanical coding and counter coding, only minor modifications have to be made to the insert and to the receptacle in order to provide a set, hence a kit of different housing for use of e.g. different drugs.

With a further example the housing comprises a groove that is provided on one of the insert and the receptacle. The groove extends along the longitudinal direction. Typically, the groove is linearly or straight shaped and extends exclusively in the longitudinal direction or parallel to the longitudinal direction. The housing further comprises a protrusion provided on the other one of the insert and the receptacle. The protrusion is configured, hence, the protrusion is sized and/or shaped to slide along the groove upon insertion of the insert into the receptacle. Mutual engagement of the groove and the protrusion rotationally locks the first housing component relative to the second housing component during the mutual assembly of the first and second housing components.

The mutual engagement of the groove and the protrusion on the insert and the receptacle provides mounting and/or mutually assembling the first housing component and the second housing component. Typically, the groove adjoins a longitudinal end face of the first connecting end or of the second connecting end. The protrusion to engage with the groove can be provided near a longitudinal end of the other one of the first connecting end and the second connecting end. In this way and right upon insertion of the insert into the receptacle the insert can be rotationally locked with respect to the receptacle. Hence, the assembly of the first and second housing components is reduced to a purely longitudinal sliding movement of the first housing component relative to the second housing component. A non-rotational assembly process, wherein the first housing component is exclusively subject to a sliding motion along the longitudinal direction relative to the second housing component is rather intuitive and easy to perform, even for patients or users suffering side effects.

According to a further example the radial recess as provided on one of the insert and the receptacle and forming a coding feature or counter coding feature respectively comprises a coded groove. The coded groove is provided on an inside surface of a sidewall of the receptacle. The coded groove adjoins a longitudinal end face of the second connecting end. Alternatively, the coded groove is provided on an outside surface of a sidewall of the insert and adjoins a longitudinal end face of the first connecting end.

The coded groove adjoins a longitudinal end face of the second connecting end or of the first connecting end. It is accessible in longitudinal direction by a complementary-shaped radial protrusion. The size or shape of the groove can define the mechanical coding or counter coding. The coded groove adjoins a longitudinal end face of the first or second connecting end. This way, inserting of a complementary shaped radial protrusion into the coded groove does not require a temporal elastic deformation of the sidewall of the receptacle for establishing a connection of the first and second housing components.

According to another example the coded groove comprises a first groove portion and a second groove portion. The first groove portion extends along the longitudinal direction and adjoins the longitudinal end face of one of the first and second connecting ends. The second groove portion extends along a circumferential or transverse direction and merges into the first groove portion at a well-defined longitudinal distance from the longitudinal end face of one of the first and second connecting ends. With some examples, wherein the coded groove is provided in a receptacle of the second housing component and wherein the coded groove is provided on an inside surface of the receptacle of the second housing component, the coded groove, in particular its first groove portion, adjoins and extends into the distal end face of the second connecting end.

The second groove portion is then provided at a longitudinal offset from the respective end face of the second housing component. The longitudinal distance between the longitudinal end face of the first or second connecting end and the longitudinal position of the second groove portion can define a coding or counter coding. Likewise, also width of the second groove portion or cross section of the first groove portion can define or contribute to at least one of a mechanical coding or mechanical counter coding.

Among a variety of different types of codings and counter coding the transverse or circumferential position of the coded groove can remain unamended. A coding can be obtained, e.g. by the width or cross-section of the second groove portion of the coded groove, by the longitudinal position of the second groove portion of the coded groove, and/or by a longitudinal distance between the second groove portion and the longitudinal end face of the respective first or second connecting ends.

According to another example the coding feature and the counter coding feature distinguish from coding features and counter coding features of another housing by varying the longitudinal position of the second groove portion and the longitudinal position of the radial protrusion correspondingly. Here, an increase of a longitudinal distance of the second groove portion from the longitudinal end face of one of the first and second connecting ends is accompanied by an increase of the longitudinal width of the second groove portion and by a corresponding increase of the longitudinal extent of the corresponding radial protrusion.

The longitudinal width of the second groove portion is the longitudinal size of the groove, hence the size or width of the groove as seen in longitudinal direction. The second groove portion can extend along the circumferential or transverse direction. Along the transverse or circumferential direction the second groove portion can comprise a constant cross-section or geometry. Typically, the first groove portion extends in longitudinal direction and the second groove portion merging the first groove portion extends along the circumferential direction.

The correspondingly shaped protrusion is intended to enter the first groove portion of the coded groove at the respective longitudinal end face of one of the first and second connecting ends. The cross-section of the protrusion in a direction transverse to the longitudinal direction and hence transverse to the direction along which the insert is inserted into the receptacle matches with the transverse width of the first groove portion of the coded groove.

When reaching an intermediate position of assembly the protrusion can align with the second groove portion. Now, when the longitudinal extent of the protrusion matches the longitudinal width of the second groove portion the protrusion is allowed to enter the second groove portion along the transverse or circumferential direction. Insofar, the insert is insertable into the receptacle by a two-stepped motion. In a first step the insert is moved along the longitudinal direction without a rotation relative to the receptacle. Here, movement of the protrusion simply follows the rather straight shaped, e.g. non-bended first groove portion.

An intermediate position can be reached when the insert gets in axial abutment with the receptacle, e.g. when a flange portion axially confining the insert axially abuts with a longitudinal end face at the free end or longitudinal end of the sidewall confining the receptacle. With other examples the intermediate position is reached when a longitudinal end of the insert axially or longitudinally abuts with an axial or longitudinal end face located inside the receptacle and confining the receptacle with regard to an insert direction.

Then and when arriving in the intermediate position the insert is subject to a rotation relative to the receptacle with the longitudinal direction as an axis of rotation. This rotating movement brings the protrusion in engagement with the second groove portion. Here, the protrusion slides along the circumferential extent of the second groove portion until a final assembly position is obtained.

Here, a coding can be provided by the longitudinal width of the second groove portion and/or by the longitudinal position of the second groove portion of the coded groove. In this regard, the first groove portion and the second groove portion are a particular example of a radial recess on one of the insert and the receptacle engaging with a radial protrusion on the other one of the insert and the receptacle.

Non-matching or mutually incompatible codings can be provided when in the intermediate assembly position, in which the insert has reached a longitudinal end position inside the receptacle the protrusion of one of the insert and the receptacle does not properly align with the second groove portion of the coded groove. Alternatively, and with other configurations it is conceivable, that upon reaching of an intermediate configuration or longitudinal end position the protrusion can at least partially align with the second groove portion but may be simply too large to enter the second groove portion. Here, the protrusion can comprise a longitudinal extent that is larger than the respective width of the second groove portion of the coded groove.

According to a further example the coding feature of the first mechanical coding distinguishes from a coding feature of another mechanical coding with regard to at least one of a number of coding features, a longitudinal position, a longitudinal extent, a circumferential position, a circumferential extent and/or by a cross-sectional geometry or shape in a plane transverse to the longitudinal direction. Likewise, and according to further examples the counter coding feature of a first mechanical counter coding distinguishes from a counter coding feature of another mechanical counter coding with regard to at least one of a number of coding features, a longitudinal position, a longitudinal extent, a circumferential position, a circumferential extent and/or a cross-sectional geometry or shape in a plane transverse to the longitudinal direction.

According to another example the fastening element comprises a snap element and the counter fastening element comprises a counter snap element complementary shaped to the snap element. The snap element is configured to engage the counter snap element to interlock the first and second housing components against at least one of a relative movement with regards to the longitudinal direction and with regards to the circumferential direction. With some examples the snap elements and the counter snap elements are operable to interlock the first and second housing component against both, a relative movement with regards to the longitudinal direction and with regard to the circumferential or transverse direction. A snap element can comprise a protrusion on one of the insert and the receptacle, while the corresponding counter snap element comprises a complementary-shaped recess on the other one of the insert and the receptacle. With some examples the snap element and the counter snap element remain unaltered with all types of codings and counter codings of the first and second housing components. Here, the snap elements and counter snap elements are provided separately from the coding feature or counter coding feature, respectively.

With other examples the mechanical coding is integrated into the snap element and is defined by a longitudinal position and one of a longitudinal extent and a transverse extent of the snap element on the insert. Correspondingly, the mechanical counter coding is integrated into the counter snap element and is defined by a longitudinal position and one of a longitudinal extent and a transverse extent of the counter snap element in the receptacle.

With other examples or when seen from another point of view the snap element can be also integrated into the mechanical coding and the counter snap element can be integrated into the mechanical counter coding. Here, the radial protrusion of the coding feature and the counter coding feature can equally serve as a radial protrusion of a snap element or counter snap element. The radial recess of the other one of the coding feature and the counter coding feature can equally serve as a counter snap feature operable to positively engage or to form a lock with the snap feature.

According to another aspect there is provided an injection device for injecting a dose of a medicament. The injection device comprises a housing as described above and a cartridge arranged inside the housing. The cartridge comprises a barrel filled with a medicament and sealed in a proximal longitudinal direction by a movable bung. The injection device further comprises a drive mechanism arranged inside the housing. The drive mechanism comprises a piston rod operable to exert a distally directed dispensing force onto the bung of the cartridge. Typically, the injection device is implemented as a hand held or portable injection device. The injection device can comprise a pen-type injector.

With some examples the receptacle is provided as a housing insert fixedly attachable or fixedly attached to an elongated housing component, e.g. the first or second housing component of the housing of the drug delivery device. The housing insert can be rotationally and/or longitudinally fixed to the elongated housing component. Insofar all features and benefits as described above in connection with the receptacle equally apply to a housing insert fixedly connectable or fixedly connected to a respective housing component.

According to another aspect, the present disclosure relates to a kit of at least a first housing as described above and at least a second housing as described above. Here, the coding feature of the first housing distinguishes from the coding feature of the second housing with regard to at least one of a number of coding features, a longitudinal position, a longitudinal extent, a circumferential position, a circumferential extent and/or a cross-sectional geometry or shape in a plane transverse to the longitudinal direction. Likewise, also the first housing comprises a first counter coding feature that distinguishes from a respective counter coding feature of the second housing with regard to at least one of the above-mentioned features, a longitudinal position, a longitudinal extent, a circumferential position, a circumferential extent and/or a cross-sectional geometry or shape in a plane transverse to the longitudinal direction.

Here, only the housing components of the first housing equipped with complementary shaped mechanical codings and mechanical counter codings are allowed and supported to become mutually fastened and fixed. The first housing is provided with a pair of a coding and counter coding of a first type. A second housing is provided with a pair of a coding and counter coding of a second type. A coding of the first type is incompatible with a counter coding of the second type. A counter coding of the first type is incompatible with a coding of the second type. A user trying to assemble a housing component of the first housing with a housing component of the second housing will be hindered to do so by not-matching mechanical codings and mechanical counter codings.

Generally, and with some examples the first housing components of different housings can distinguish by the size and/or geometry of an accommodating space for receiving a medicament container or cartridge. In particular, a housing with a coding of a first type can be exclusively equipped with a first cartridge or medicament container. A housing with a coding of a second type can be exclusively equipped with a cartridge or a second medicament container. For this, medicament containers, cartridges as well as the interior of the first housing components can comprise further codings or coding features or can distinguish with regard to their size or geometry such that only one dedicated cartridge or medicament container unequivocally fits into only one dedicated first housing component.

With some examples, the first housing component is provided with a mechanical coding to engage with a complementary shaped counter coding of a cartridge. With further examples the first housing component can be provided with at least one of an electronic, a visual or optical coding configured to match with a complementary counter coding of the cartridge, which is also of electronic, visual or optical type.

Moreover, at least one of the cartridge and the first housing component can be provided with a locking or fastening feature by way of which a cartridge can be fixed and/or retained in the first housing component. Here, the first housing component, e.g. implemented as a cartridge holder, and a cartridge assembled therein can be provided as a pre-fabricated housing assembly or as a dedicated cartridge-cartridge holder combination.

In either way, it can be assured or provided that a particular medicament provided in a particular cartridge is unequivocally associated with a particular type of a first housing component, i.e. with a particularly mechanically encoded first housing component. In effect and with some examples, a cartridge provided with a particular medicament, for example, can be only accommodated in a correspondingly shaped first housing component equipped with a respective mechanical coding.

With further examples a pre-fabricated housing assembly or a dedicated cartridge-cartridge holder combination is commercially distributed by a pharmaceutical manufacturer. Here, the cartridge can be undetachably or irremovably fixed inside the first housing component and the pharmaceutical manufacturer provides a respective matching between a cartridge filled with a particular medicament and a suitable first housing component, which is mechanically encoded in accordance to the type of medicament located inside the cartridge.

According to a further aspect the present disclosure also relates to a kit of injection devices. The kit of injection devices comprises at least a first injection device comprising a first housing provided with a coding and a counter coding both of a first type and further comprises a second injection device with a second housing provided and equipped with a coding and a counter coding both of a second type non-matching with the respective counter coding or coding of the first type.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament can be used for a limited duration, or on a regular basis for chronic disorders.

As described below, a drug or medicament can include at least one API, or combinations thereof, in various types of formulations, for the treatment of one or more diseases. Examples of API can include small molecules having a molecular weight of 500 Dalton (Da) or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament can be contained in a primary package or “drug container” adapted for use with a drug delivery device. The drug container can be, e.g., a cartridge, syringe, reservoir, or other solid or flexible vessel configured to provide a suitable chamber for storage (e.g., short- or long-term storage) of one or more drugs. For example, in some instances, the chamber can be designed to store a drug for at least one day (e.g., 1 to at least 30 days). In some instances, the chamber can be designed to store a drug for about 1 month to about 2 years. Storage can occur at room temperature (e.g., about 20° C.), or refrigerated temperatures (e.g., from about −4° C. to about 4° C.). In some instances, the drug container can be or can include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge can be configured to allow mixing between the two or more components prior to and/or during dispensing into the human or animal body. For example, the two chambers can be configured such that they are in fluid communication with each other (e.g., by way of a conduit between the two chambers) and allow mixing of the two components when desired by a user prior to dispensing. Alternatively or in addition, the two chambers can be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of injection devices with dedicated or coded housing components will be described in greater detail by making reference to the drawings, in which:

FIG. 1 schematically illustrates an example of a drug delivery device,

FIG. 2 shows an example of an exploded view of the drug delivery device of FIG. 1,

FIG. 3 shows an example of a coded housing of a drug delivery device,

FIG. 4 shows another example of a coded housing,

FIG. 5 shows a further example of a coded housing,

FIG. 6 is a perspective illustration of a first connecting end of a first housing component,

FIG. 7 is a cross-sectional illustration of the first connecting end connected to the second connecting end of a second housing components,

FIG. 8 is an example of a coding feature and a complementary-shaped counter coding feature of a first type,

FIG. 9 is another example of a mechanical coding and a mechanical counter coding of a second type,

FIG. 10 is another example of a mechanical coding and a mechanical counter coding of a third type,

FIG. 11 is an example of a coding feature and a complementary-shaped counter coding feature of a first type,

FIG. 12 is another example of a mechanical coding and a mechanical counter coding of a second type,

FIG. 13 is another example of a mechanical coding and a mechanical counter coding of a third type,

FIG. 14 is illustrative of a further example of a mechanical coding provided on a first housing component,

FIG. 15 is illustrative of a complementary-shaped counter coding provided in a receptacle of a second housing component,

FIG. 16 shows a further example of a mechanical coding,

FIG. 17 shows a further example of a mechanical counter coding,

FIG. 18 is a longitudinal cross-section through a first housing component and a second housing component according to FIGS. 16 and 17,

FIG. 19 is a perspective cross-sectional view of the second housing component according to FIG. 17,

FIG. 20 shows another example of a mechanical coding of a first housing component,

FIG. 21 is illustrative of a further counter coding of a second housing component,

FIG. 22 is illustrative of a longitudinal cross-section through mutually coded first and second housing components,

FIG. 23 is a perspective and partial cross-section through the mechanical counter coding of FIG. 21,

FIG. 24 shows another example of a mechanical coding,

FIG. 25 shows a further example of a mechanical counter coding,

FIG. 26 shows another example of a mechanical coding and a complementary shaped mechanical counter coding of a first type,

FIG. 27 is illustrative of a mechanical coding and a mechanical counter coding of a second type,

FIG. 28 shows another example of a mechanical coding and mechanical counter coding of a third type.

FIG. 29 is an enlarged view of the proximal connecting end of the first housing component, and

FIG. 30 is a detailed view of the receptacle as provided in the second housing component of FIG. 29.

### DETAILED DESCRIPTION

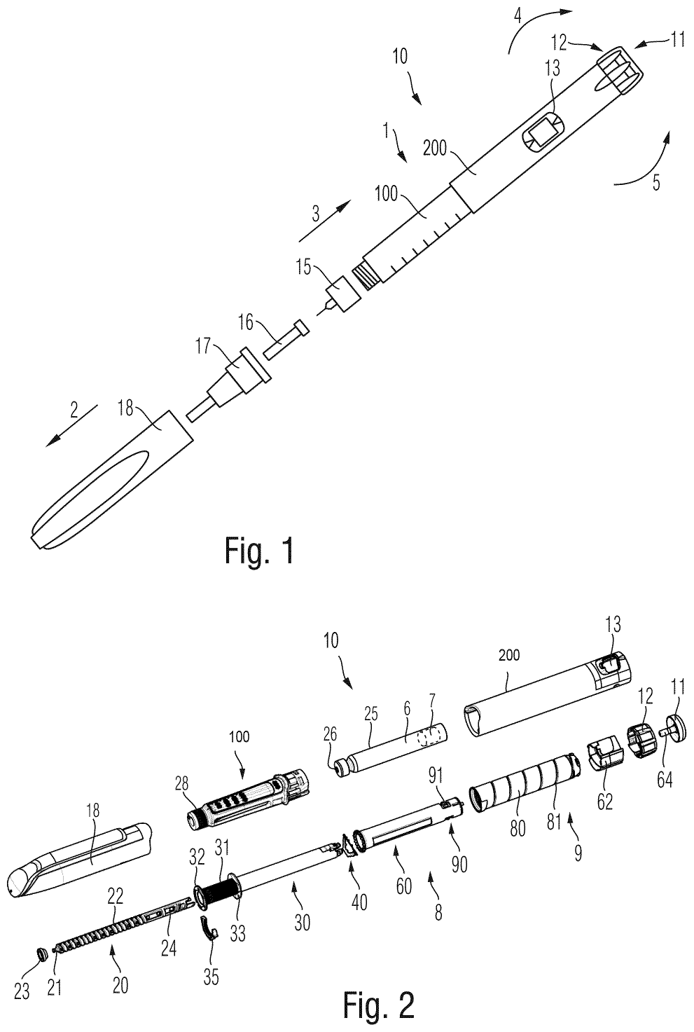

In FIGS. 1 and 2, one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component can form a main housing part configured to accommodate a drive mechanism 8 and/or a dose setting mechanism 9 as shown in FIG. 2. The first housing component 100 is configured as a cartridge holder. It can be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 can be displayed in so-called international units (IU, wherein 1 IU is the biological equivalent of about 45.5 μg of pure crystalline insulin ( 1/22 mg). The dose dial 12 can comprise or can form a dose dial.

As shown further in FIGS. 1 and 2, the housing 10, e.g. the second housing component 200 comprises a dosage window 13 that can be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 can be configured so that turning the dose dial 12 (e.g., a dosage knob) causes a mechanical click sound to provide acoustical feedback to a user. The click sound is typically generated by a click noise generator 45. Generally, a click noise generator 45 can be implemented in various different ways. The number sleeve 80 mechanically interacts with a piston in the insulin cartridge 6. When the needle 15 is stuck into a skin portion of a patient, and when the trigger 11 or injection button is pushed, the dose displayed in dosage window 13 (e.g., display window) will be ejected from injection device 1. When the needle 15 of the injection device 1 remains for a certain time in the skin portion after the trigger 11 is pushed, the dose is actually injected into the patient's body. Ejection of a dose of the liquid medicament can also cause a mechanical click sound, which is however different from the click sound produced when using the dose dial 12. For this, the injection device one can comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 can be used for several injection processes until either the cartridge 6 is empty or the expiration date of the medicament in the injection device 1 (e.g. 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g. to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number sleeve 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number sleeve 80 to prevent relative movement there between. The dose dial 12 is provided with a central opening.

The trigger 11, also denoted as dose button is substantially T-shaped. It is provided at a proximal end of the injection device 1. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring 40 or clicker then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch can serve as a ratchet mechanism.

As an alternative or in addition the ratchet mechanism 90 can comprise at least one ratchet feature 91, such as a flexible arm on the sidewall of the tubular-shaped clutch 60. The at least one ratchet feature 91 can comprise a radially outwardly extending protrusion e.g. on a free end of the flexible arm. The protrusion is configured to engage with a correspondingly shaped counter ratchet structure on an inside of the number sleeve 80. The inside of the number sleeve 80 can comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user can simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position can be determined by the abutment of one of axially extending edges or stops of the number sleeve 80 with at least one or several corresponding stops of the housing 10.

The expelling mechanism or drive mechanism 8 as described above is exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

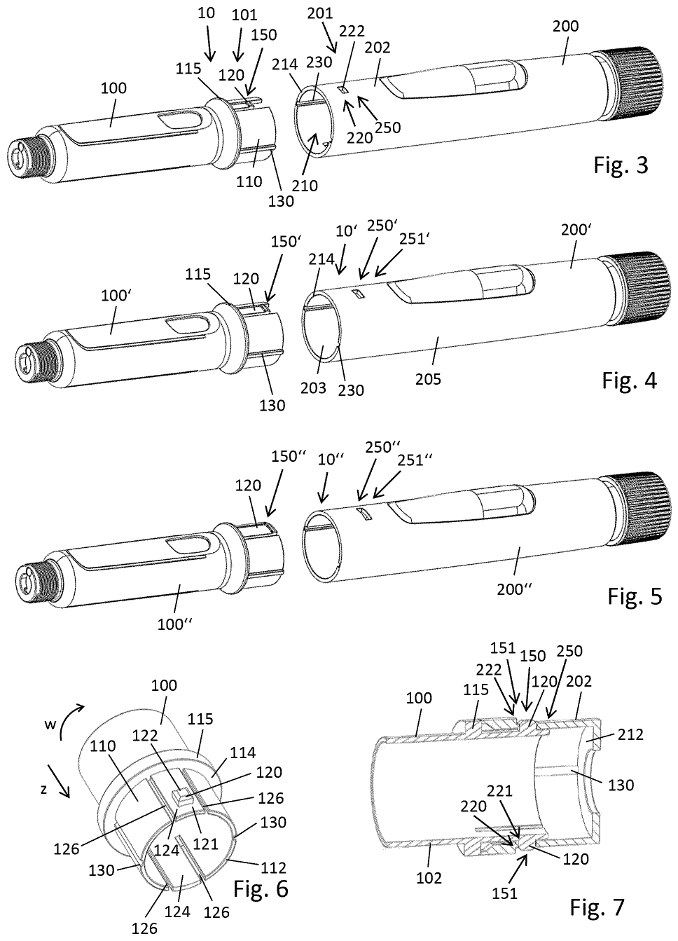

The housing 10 as illustrated in any of the FIGS. 3-30 comprises a first housing component 100 and a second housing component 200. The first housing component 100 is configured as a cartridge holder. It is sized and shaped to accommodate a cartridge 6 inside its hollow interior. The cartridge holder and hence the first housing component 100 comprises a first connecting end 101. The first connecting end 101 forms a proximal end of the first housing component 100. Correspondingly, the second housing components 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The insert 110 is insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110.

The flange section 115 comprises a circumferential rim extending all around the tubular shaped insert 110. Towards the proximal direction 3 the flange section 115 comprises an abutment face 114 facing in proximal direction 3. The abutment faces 114 is configured to axially abut a distal end face 214 of the sidewall 202 of the second housing component.

For mutually fixing the first and second housing components 100, 200 there is provided a fastening element 120 on the insert 110 to operably engage with a correspondingly or complementary-shaped counter fastening element 220 provided inside the receptacle 210. In the presently illustrated examples, as for instance shown in greater detail in FIGS. 6 and 7 the fastening element 120 comprises a snap element 121 configured to engage with the correspondingly or complementary shaped counter snap element 221 as provided on an inside surface 203 of the sidewall 202 of the receptacle 210. The snap element 121 comprises a radial protrusion 122 as illustrated in FIG. 6 shaped and configured to engage with radial recess 222 of the counter fastening element 220 on the sidewall 202 of the receptacle 210.

As shown in FIG. 7, there are provided numerous fastening elements 120 and complementary-shaped counter fastening elements 220 on the outside surface of the sidewall 102 of the insert 110 and on the inside surface 203 of the sidewall 202 of the receptacle 210, respectively. By way of mutually corresponding snap elements 121 and counter snap elements 221 a snap-fit engagement of the first and second housing components 100, 200 can be provided.

On the outside surface 105 of the insert 110 there is further provided a longitudinal protrusion 130 or rib extending from the longitudinal end face 112 to the flange section 115. In the presently illustrated example, there are provided two diametrically oppositely located longitudinal grooves 230. On the inside surface 203 of the receptacle 210 there is provided at least one longitudinal groove 230 that is shaped and sized to engage with and to slide along the protrusion 130 upon insertion of the insert 110 into the receptacle 210. The protrusion 130 and the groove 230 provide a keyed engagement of the insert 110 and the receptacle 210. By way of the keyed engagement the insert 110 is rotationally locked to the receptacle 210. Thus, a mutual fixing and fastening of the first housing component 100 and the second housing components 200 is obtained by a purely longitudinal sliding motion of the insert 110 into the receptacle 210.

In FIGS. 3-7 an example of a mechanical coding 150 complementary shaped to a mechanical counter coding 250 is schematically illustrated. As shown in greater detail in FIG. 6 the insert 110 of the first housing component 100 comprises a fastening element 120 featuring a snap element 121 configured to engage with a complementary-shaped counter snap element 221 of a counter fastening element 220 as provided on the second housing component 200. The fastening element 120 is implemented as a snap element 121. It comprises a radial protrusion 122 or projection complementary shaped to a radial recess 222 in the sidewall 202 of the second housing component 200. The radial recess 222 is presently illustrated as a through recess extending entirely through the sidewall 202. With other examples the radial recess 222 is a blind recess only provided on the inside surface of 203 of the receptacle 210. Here, the outside surface 205 of the second housing components 200 is void of any recesses or the like counter fastening elements 220.

In the illustrated example the radial protrusion 122 of the snap element 121 protrudes radially outwardly from the insert 110. It can be elastically deformable in radial direction. This can be achieved by providing the radially outwardly extending radial protrusion 122 on the outside surface of a tongue portion 124. The tongue portion 124 is a part of the insert 110 but is separated from the sidewall of the insert 110 by a first and a second longitudinal slit 126 confining the tongue portion 124 in circumferential direction (w). The slits 126 extend in longitudinal direction (z) from the flange section 115 towards the longitudinal end face 212. The tongue portion 124 can be exclusively connected to the flange section 115. It can be elastically bendable, elastically deformable and/or pivotably supported on the insert 110 with regard to the radial direction.

At a longitudinal end of the tongue portion 124 facing towards the insert direction and hence facing towards the free end of the respective housing component 100 there is provided a beveled section or chamfer 125. This beveled section or chamfer 125 induces and supports a radially inwardly directed bending or flexing of the tongue portion 124 when getting in contact with the sidewall 202 of the receptacle 210.

In the example of FIGS. 6 and 7, it is the insert 110 that comprises a longitudinal groove 230 configured to engage with a longitudinally extending protrusion 130 extending radially inwardly from the sidewall 202 of the receptacle 210.

The fastening element 120 is integrally formed with the mechanical coding 150. In other words, the mechanical coding 150 can be integrated into the fastening element 120; and vice versa. Accordingly, the protrusion 122 forms or constitutes the mechanical coding feature 151. Accordingly, the radial recess 222 as provided in the sidewall 202 of the receptacle 210 defines the mechanical counter coding 250. At least one of the geometry, the geometric extent and/or the position of the radial protrusion 122 defines the mechanical coding feature 151. Likewise, at least one of the geometry, the extent, the size and/or the position of the radial recess 222 defines the mechanical counter coding features 251.

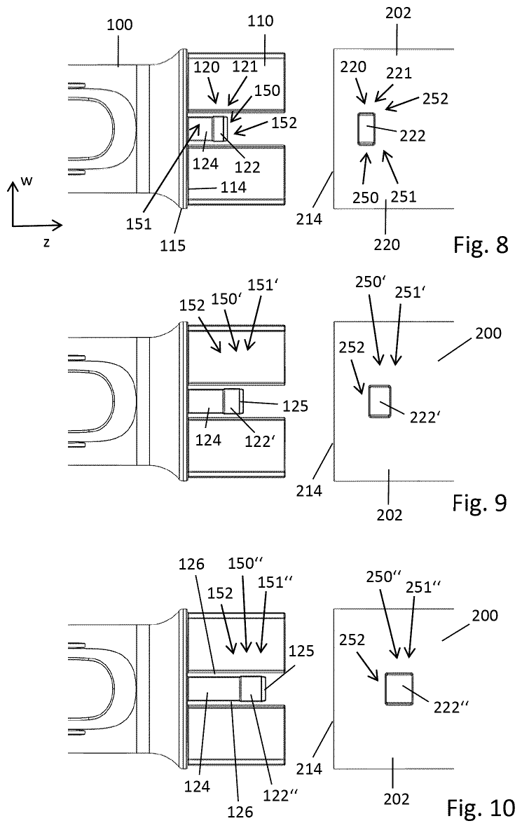

In the examples as illustrated in the sequence of FIGS. 8-10 there are illustrated three different pairings of mutually corresponding mechanical codings 150, 150′,150″ and respective mechanical counter codings 250, 250′, 250″. Insofar, each one of the FIGS. 8-10 illustrates a particularly encoded housing 10, 10′, 10″ of a drug delivery device 1. A first housing component 100 of a housing 10 cannot engage with a second housing component 200′, 200″ of any of the other housings 10′, 10″. Vice versa, a second housing component 200 of the housing 10 cannot engage with a first housing component 100′, 100″ of any one of the other housings 10′, 10″.

Insofar, FIGS. 8-10 are illustrative of a kit of numerous housings and 10, 10′, 10″ of injection devices, wherein a first housing component 100 of a selected housing 10, 10′, 10″ is exclusively connectable to a second housing component 200 of the same housing 10 and is prevented from connecting with a second housing component 200′, 200″ of any of the other housings 10′, 10″ of the kit of housings.

The mechanical coding feature 151 is defined by a combination of the longitudinal position of the fastening element 120 and hence by a combination of the longitudinal position of the respective snap element 121 and the longitudinal extent of the snap element 121 and/or of its radial protrusion 122. Generally, the snap element 121 can be regarded as a coding feature 151 and the radial protrusion 122 can be regarded as a radial coding protrusion 152.

As can be seen in the sequence of FIGS. 8-10 the longitudinal position the coding feature 151, 151′, 151″ varies with regard to the longitudinal direction (z). In the same way also the longitudinal extent of the coding feature, in particular the longitudinal extent of the radial protrusion 122, 122′, 122″ varies. In the same way also the longitudinal position of the complementary shaped counter coding features 251, 251′, 251″ and the respective longitudinal extent of the radial recess 222, 222′, 222″ varies accordingly. In this way it can be ensured, that the mechanical coding 150 is exclusively pairable, combinable and/or engageable with the mechanical counter coding 250.

In a final assembly configuration, i.e. when the proximal face 114 of the flange section 115 axially abuts with the distal end face 214 of the second housing component 200 the radial protrusion 122 of the snap element 121 engages the radial recess 222 of the complementary shaped counter fastening element 220. As illustrated, the longitudinal extent of the radial protrusion 122 closely matches the longitudinal extent of the radial recess 222. As the longitudinal extent of the radial protrusion 122′, 122″ gradually increases as it is apparent by the examples of FIGS. 9 and 10 the longitudinal position of the respective coding feature 151′, 151″ separates more and more from the abutment face 114.

Accordingly, the longitudinal distance of the radial recess 222 and hence of the counter coding features 251′, 251″ separates more and more from the end face 214.

Accordingly, by varying the longitudinal position of the radial recess 222, 222′, 222″ and the longitudinal position of the radial protrusion 122, 122′, 122″ correspondingly, an increase of a longitudinal distance of the radial recess 222, 222′, 222″ on a free end of the second connecting end 201 is accompanied by an increase of the longitudinal extent of the radial recess 222, 222′, 222″ and by a corresponding increase of the longitudinal extent of the correspondingly shaped radial protrusion 122, 122′, 122″. In certain implementations, the coding feature 151 is exclusively engageable with only one particular counter coding feature 251 of the available counter coding features 251, 251′, 251″.

In an attempt that the coding feature 151 would be paired with one of the non-matching counter coding features 251′, 251″, hence when attempting to engage the coding feature 151 of the insert 110 of FIG. 8 into the receptacle 210 provided with the counter coding features 251′, 255′ as shown in FIG. 9 or 10 the radial protrusion 122 will not reach the longitudinal position of the respective counter coding features 251′, 255″. Before spatially overlapping with any of the counter coding features 251′, 251″ the abutment face 114 engages the axial end face 214 and blocks any further proximally directed movement of the insert 110 into the receptacle 210.

The other way round and when attempting to insert any of the coding feature 151′, 151″ of any of the FIG. 9 or 10 into the receptacle 210 of FIG. 8 the radial protrusion 122′, 122″ can slide over and across the radial recess 222. But since the longitudinal extent of the radial protrusions 122′, 122″ is larger than the longitudinal extent of the radial recess 222 the radial protrusions 122′, 122″ cannot engage the radial recess 222. The longitudinal extent of the recess 222 is smaller than the longitudinal extent of the radial protrusions 122′, 122″. Accordingly, the coding features 151′, 151″ are hindered to engage the counter coding feature 251. Due to this difference with regard to the longitudinal extent the snap elements 121′, 121″ cannot engage the counter snap element 221.

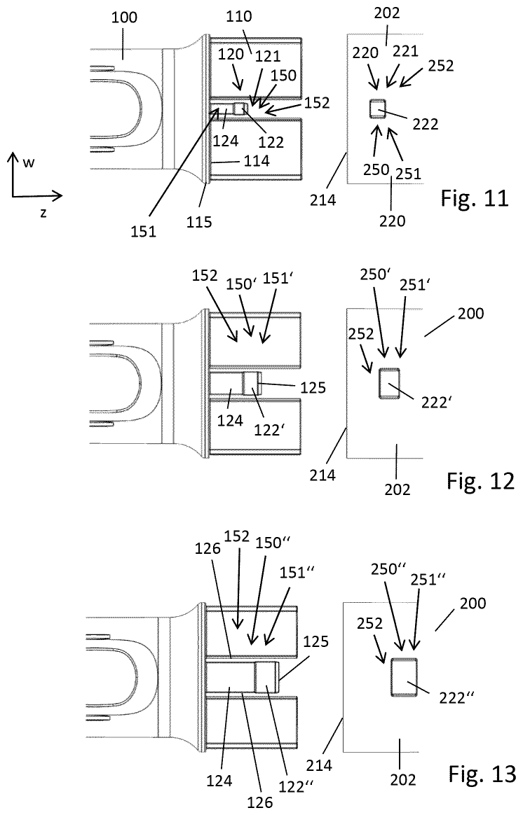

In the further illustration of the sequence of FIGS. 11-13, another example of a mechanical coding 150, 150′, 150″ of a kit of housings 10, 10′, 10″ is illustrated. Here, not only the longitudinal position and/or the longitudinal extent of the snap element 121, 121′, 121″ is subject to a modification and geometric variation but also the extent of the respective snap element 121, 121′, 121″ with regards to the circumferential extend, hence along the circumferential direction (w) is subject to a respective modification.

The size and position of the complementary shaped counter codings 250, 250′, 250″ and respective counter coding features 251, 251′, 251″ is subject to a respective modification and variation. As becomes apparent from the illustration of the sequence of FIGS. 11-13 the radial protrusion 122′ is located further away in longitudinal direction from the abutment face 214 than the radial protrusion 122. The same applies to the radial recess 222′ in relation to the radial recess 222 with regards to the longitudinal end face 214. At the same time the longitudinal extent and the circumferential extent of the radial protrusion 122′ is larger than the longitudinal extent and the circumferential extent of the radial protrusion 122.

The same applies by a comparison of the radial protrusion 122″ with regards to the radial protrusion 122′. The circumferential and longitudinal extent or size of the radial protrusion 122″ is larger than the circumferential and longitudinal extend or size of the radial protrusion 122′. In this way and when attempting to engage one of the mechanical coding 150′, 150″ or one of the coding features 151′, 151″ with the mechanical counter coding 250 or mechanical counter coding feature 251 the respective radial protrusion 122′, 122″ fails to engage with the counter coding features 251 or radial recess 222 because the circumferential and longitudinal extent of the radial recess 222 is smaller than the circumferential or longitudinal extent of any of the radial protrusions 122′, 122″.

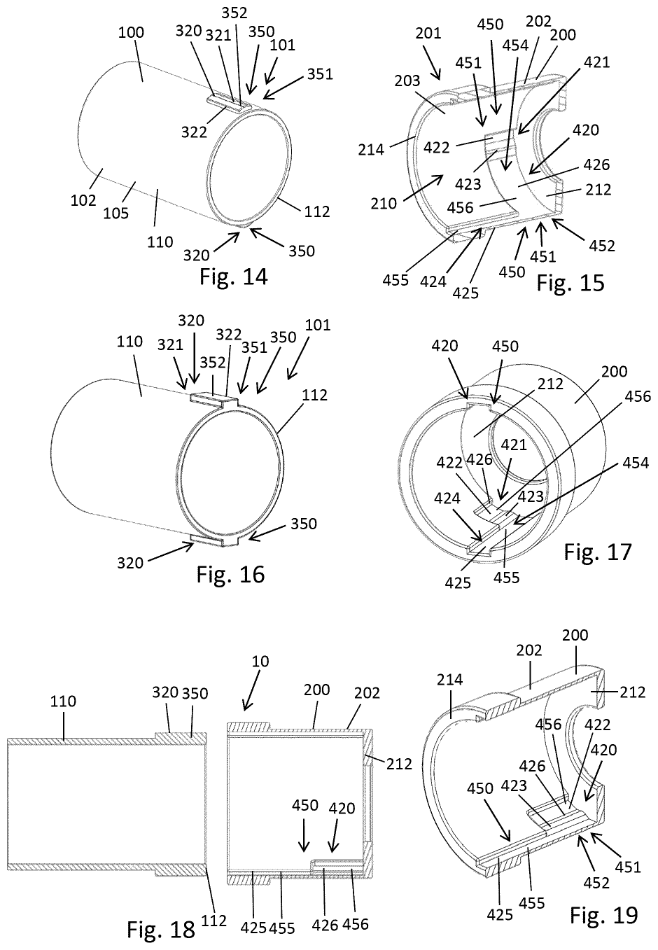

In FIGS. 14-19 an example of a mechanical coding 350 complementary shaped to a mechanical counter coding 450 is illustrated. The mechanical coding 350 comprises a mechanical coding feature 351. The coding feature 351 comprises a radial protrusion 352 provided on an outside surface 105 of the insert 110 at the first connecting end 101 of the first housing component 100. Also here, the first housing component 100 comprises a tubular-shaped sidewall 102. Towards a proximal direction the sidewall 102 comprises a proximal end face 112. The proximal end face 112 is planar shaped and comprises an annular structure. In the illustrated example the mechanical coding 350 is provided twice on the outside surface 105 of the sidewall 102 of the first housing component 100. It is provided at a particular circumferential position and at a diametrically oppositely located position. Such a twofold coding 350 is beneficial to enhance the stability and mechanical rigidity of the mechanical coding 350 engaging with a complementary shaped mechanical counter coding 454.

In the illustrated example the fastening element 320 is integrated into the mechanical coding feature 351. Here, the fastening element 320 comprises a snap element 321. The fastening element 320 comprises a radial protrusion 322 protruding radially outwardly on the outside surface 105 of the sidewall 102. The radial protrusion 322 comprises an elongated rib aligned along the longitudinal direction. In the illustrated example the radial protrusion 322 adjoins the end face 112.

The counter coding feature 451 as illustrated in FIGS. 15 and 19 comprises a radial recess 452 on an inside surface of the sidewall 202 of the receptacle 210. The counter coding feature 451 of the counter coding 450 comprises a radial recess 452. The radial recess 452 comprises a coded groove 454. The coded groove 454 comprises a first groove portion 455 and a second groove portion 456. The first groove portion 455 adjoins a distal end face 412 of the sidewall 202 of the second housing component 200.

The first groove portion 455 extends in longitudinal direction. It is rather straight shaped and extends from the end face 214 towards the oppositely located end face 212 formed by a radially inwardly extending flange portion of the sidewall 202 and delimiting the receptacle 210 towards the proximal direction 3. The second groove portion 456 extends in circumferential direction (w). The second groove portion 456 merges into the first groove portion 455. The first groove portion 455 and the second groove portion 456 form a L-shaped coded groove 454. In the illustrated example the second groove portion 456 adjoins the distal side of the end face 212.

The coded groove 454 coincides with a groove 424 of the counter fastening element 420. In other words, the counter fastening element 420 and the counter coding 450 share the same geometric structure on the inside surface 203 of the receptacle 210. In other words, the counter fastening element 420 is integrated into the mechanical counter coding 450. Vice versa, the mechanical counter coding 450 is integrated into the counter fastening element 420. In this way, the counter fastening element 420 comprises a counter snap element 421 to engage with the snap element 321 of the fastening element 320. The counter fastening element 420 comprises a raised ridge 423 and a recess 422. The recess 422 and the raised ridge 423 constitute or form the counter snap element 421 to engage with the snap element 321 of the fastening elements 320.

As illustrated the raised ridge 423 comprises a radially inwardly extending protrusion at or near a dead end of the second groove portion 426. Here, the second groove portion 426 coincides with the second groove portion 456. A first groove portion 425 coincides with the first groove portion 455.

For connecting the first connecting end 101 to the second connecting end 201 the insert 110 is inserted into the receptacle 210 along the longitudinal direction. In order to enable a longitudinal sliding insert motion of the insert 110 into the receptacle 210 the fastening element 320 and hence the mechanical coding feature 351 has to be aligned with the complementary-shaped counter fastening element 420 and the respective mechanical counter coding feature 451, respectively. When appropriately aligned, the radial protrusion 322, 352 can be inserted into the first groove portion 425, 455. The insert 110 is then allowed to move in longitudinal direction relative to the second housing component 200 until the longitudinal end face 112 get in axial abutment with the end face 212 of the receptacle.

Then, an intermediate assembly configuration has been reached. If appropriately encoded, hence if the mechanical coding 350 matches the counter coding 450, the coding feature 351 matches with the counter coding feature 451 with regards to its longitudinal position as well as with regard to its longitudinal extend. Then in the intermediate assembly configuration the radial protrusion 322, 352 is longitudinally aligned with the second groove portion 426, 456 and the longitudinal extent of the protrusion 322, 352 matches a longitudinal width of the second groove portion 426, 456.

Then and in a second step of assembly the first housing component 100 and hence the insert 110 is allowed to be rotated clockwise relative to the receptacle 210 with a longitudinal central axis of the housing component 100, 200 as an axis of rotation. As a consequence, the radial protrusion 322, 352 enters the second groove portion 426, 456 until the radial protrusion 322 and hence the snap element 321 positively engages with the counter snap element 421.

Finally, and when reaching a final assembly configuration the protrusion 322, 352 is located in the radial recess 422 at the dead end of the second groove portion 426, 456, which is opposite to that particular end of the second groove portion 426, 456 that merges into the first groove portion 425, 455.

With the example of FIGS. 16-19 the circumferential extent of the second groove portion 426, 456 is somewhat shorter compared to the example of FIG. 15. Here, the counter snap element 421 formed by the radially inwardly protruding ridge 423 and the radial recess 422 is located circumferentially adjacent to the first groove portion 425, 455. Also here, a mechanical coding 350 matches with a mechanical counter coding 450 only if a longitudinal extent and a longitudinal position of the radial protrusion 352 matches, i.e. spatially overlaps with the size and position of the second groove portion 426, 456.

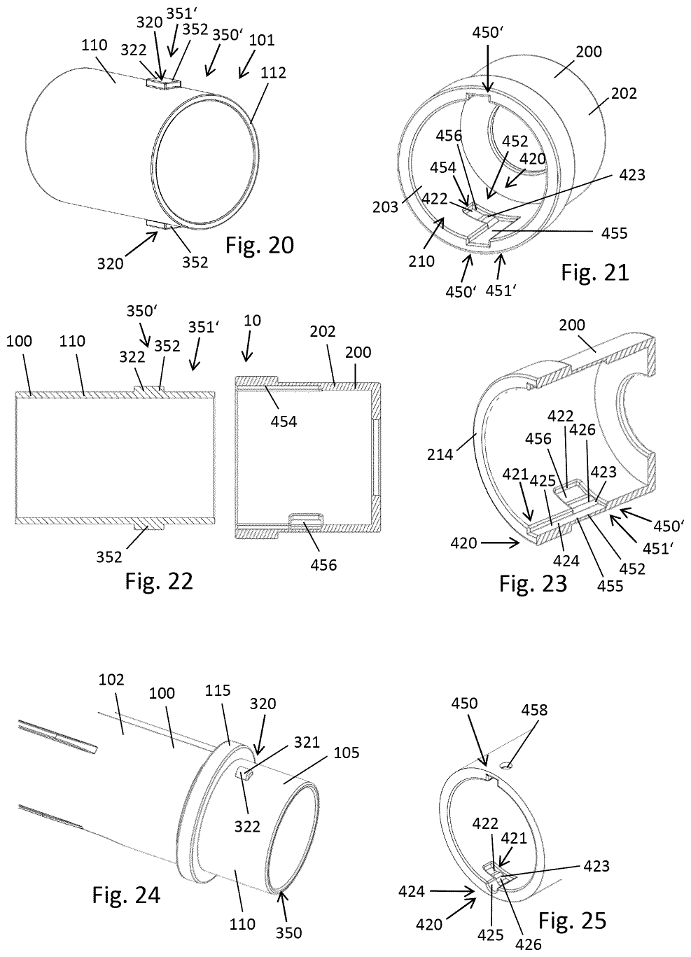

In FIGS. 20-23 another example of a coding feature 351′ and a complementary-shaped counter coding feature 451′ is illustrated. The respective coding features and counter coding features 351′ and 451′ represent a pair of a coding and counter coding of a second type. Here, the coding feature 351′ also comprises a radial protrusion 352 complementary shaped to a radial recess 452 provided on the inside surface 203 of the sidewall 202 of the receptacle 210. Contrary to the coding 350 and counter coding 450 as shown in FIGS. 14-19 the coding feature 351 and hence the protrusion 352 is located at a longitudinal offset from the longitudinal end face 112 of the insert 110. Correspondingly, the counter coding feature 451′ in the form of the coded groove 455 comprises a coded groove 454 that distinguishes from the coded groove as illustrated in FIGS. 14-19. As shown in FIGS. 21-23 the second groove portion 456 of the counter coding feature 451′ is located at a predefined distance from the end face 212.

The second groove portion 426, 456 of the counter fastening element 420 and of the counter coding 450′ is located at a distance from the longitudinal end face 214 that is smaller than the respective distance as shown in the example of FIGS. 14-19. In addition, also the longitudinal extent of the second groove portion 426, 456 has been reduced compared to the second groove portion 426, 456 of the counter fastening element 420 or counter coding feature 451 of the first type as shown in FIGS. 14-19.

Apart from that the overall geometry of the receptacle 210 and of the insert 110 have remain substantially unamended and the cross-section of the groove 455 of the counter coding feature 451′ of the second type is identical to the cross-section of the first groove portion 455 of the counter coding feature 451 of the first type.

The coding 350 and counter coding 450 of a first type as illustrated in FIGS. 14-19 is incompatible with the coding 350′ and the complementary-shaped counter coding 450 as illustrated in FIGS. 20-23. If a user should attempt to engage the coding 350 as shown in FIG. 14 or 16 into a non-matching or incompatible counter coding 450′ the respective coding protrusion 352 of the coding 350 can align with the second coding grooves 456 of the counter coding 450′. Since the longitudinal extent of the radial protrusion 352 of the coding 350 is larger than the longitudinal width of the groove portion 456 of the counter coding 450′ the coding 350 of the first type cannot engage with a groove portion 456 of a mechanical counter coding 450′ of the second type.

Vice versa, the mechanical coding 350′ of the second type is incompatible with the mechanical counter coding 450 of the first type. It may be possible to insert the insert 110 as illustrated in FIG. 20 into a receptacle 210 of FIG. 15 or 17. When reaching the intermediate assembly configuration, i.e. when the end face 112 longitudinally abuts the end face 212, the radial protrusion 352 of the mechanical coding 350′ is not in alignment with the second groove portion 426 of the mechanical counter coding 450. The protrusion 352 of the mechanical coding 350′ is longitudinally offset from the second groove portion 456 of the mechanical counter coding 450 of the first type.

Accordingly, a rotation of the insert 110 relative to the receptacle 210 with the longitudinal axis as an axis of rotation is effectively blocked and impeded. The non-matching or in compatible coding and counter coding of first and second types prevent and block an engagement of the fastening element 320 engaging the counter fastening element 420.

As a general rule and for establishing a kit of at least a first housing 10 and a second housing 10′ it is provided that an increase of a longitudinal distance of a radial recess 452, hence the longitudinal position of the second groove portion 426, 456 from a free end of the respective connecting end 201 is accompanied by an increase of the longitudinal extent of the radial recess and buy a corresponding increase of the distance and the longitudinal extent of the correspondingly shaped radial protrusion 352. The larger the longitudinal extent of the protrusion 352 the larger should be the distance of the radial protrusion 352 from a free end of the respective connecting end 101.

In FIGS. 24 and 25 another example of a fastening element 320 and of a mechanical coding is illustrated. Here, the fastening element 320 and the mechanical coding 350 do not coincide or spatially overlap. Rather, the fastening element 320 is provided at a particular circumferential position on the outside surface 105 of the sidewall 102 of the insert 110. The coding 350 is provided at a different circumferential position on the outside surface 105 of the insert 110. The fastening element 320 comprises a protrusion 322 protruding radially outwardly from the outside surface 105 of the insert 110. As illustrated in FIG. 25 the protrusion 322 is configured for insertion into a complementary-shaped counter fastening element 420.

The fastening element 320 and the rather convex-shaped radial protrusion 322 form a snap element 321. The counter fastening element 420 comprises a complementary shaped counter snap element 421. The counter snap element 421 comprises a radially inwardly extending projection or ridge 423 and a radial recess 422 located circumferentially adjacent the radially raised ridge 423. The ridge 423 and the recess 422 are located in a second groove portion 426 extending in circumferential direction.

The counter fastening element 420 comprises a first groove portion 425 and a second groove portion 426. The first groove portion 425 extends in longitudinal direction and the second groove portion 426 extends in circumferential direction. The second groove portion 426 merges into the first groove portion 425.

As shown in FIG. 25, the cross-sectional geometry of the first groove portion 425 matches the convex shape of the radial protrusion 322.

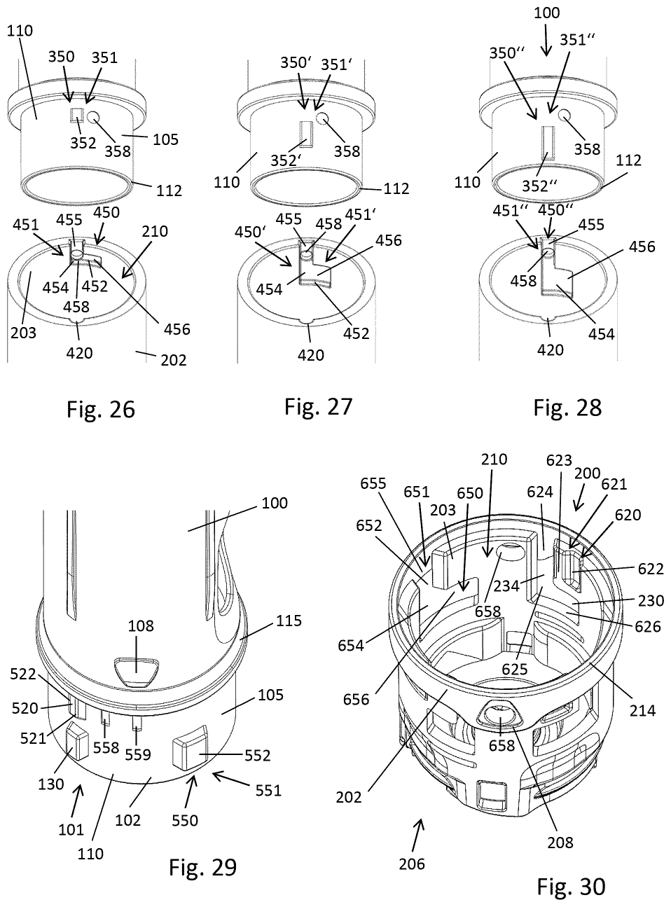

Circumferentially offset, e.g. diametrically opposite to the counter fastening element 420 there is provided the counter coding 450. Numerous examples of different codings 350, 350′, 350″ and correspondingly shaped counter codings 450, 450′, 450″ are illustrated in the sequence of FIGS. 26-28.

As becomes apparent from a comparison of the number of codings 350, 350′, 350″ the longitudinal position of a coding protrusion 352, 352′, 352″ changes together with the longitudinal extent of the respective radial protrusion 352, 352′, 352″. As can be seen by a comparison of the coding 350 of a first type with the coding 350′ of a second type the longitudinal extent of the protrusion 352′ is larger than the longitudinal extent of the protrusion 352.

Also, the longitudinal position and hence the longitudinal distance between the radial protrusion 352′ to the longitudinal end face 112 is shorter with the second type of a coding 350′ than a longitudinal distance between the protrusion 352 and the end face 112 with the first type of a coding 350. In the same way and when comparing the coding 350′ and the respective coding feature 351′ with the coding 350″ and the respective coding features 351′, respectively, the longitudinal extent of the radial protrusion 352″ is larger than the longitudinal extent of the protrusion 352′. Moreover, the longitudinal position of the radial protrusion 352″ is shifted towards the longitudinal end face 112 compared to the longitudinal position of the radial protrusion 352′.

The complementary-shaped counter codings 450, 450′, 450″ and the respective counter coding features 451, 451′, 451″ are subject to respective modifications.

With all examples the counter coding feature 451, 451′, 451″ comprises a coded groove 455 on the inside surface 203 of the receptacle 210. The coded groove 454 comprises a longitudinally extending first groove portion 455 and a circumferentially extending second groove portion 456 merging into the first groove portion 455. The first sections of the first groove portion 455 of the counter coding features 451, 451′, 451″ remain unamended are equal with all counter coding features 451, 451′, 451″.

The coding feature 451′ distinguishes from the coding feature 451 in that the longitudinal extent of the first groove portion 455 is larger. Moreover, the longitudinal extent of the first groove portion 455 of the counter coding feature 451″ of the third type is larger than the longitudinal extent of the first groove portion 455 of the counter coding feature 451′ of the second type.

The longitudinal width and hence the longitudinal extent of the second groove portion 456 varies with the counter coding features 451, 451′, 451″. The longitudinal width of the second groove portion 456 of the counter coding feature 451″ is larger than the longitudinal width of the second groove portion 456 of the counter coding feature 451′. The longitudinal width of the second groove portion 456 of the counter coding feature 451′ is larger than the longitudinal width of the second groove portion 456 of the counter coding feature 451. In this way it can be provided and ensured, that the coding 350 only and exclusively matches the counter coding 450. It cannot engage or cooperate with any of the further counter codings 450′, 450″ or counter coding features 451′, 451″.

Likewise, the coding 350′ of the second type is incompatible with any of the mechanical counter coding 450 of the first type or the mechanical counter coding 450″ of the second type. The same is valid for the coding feature 351″ of the second type. The coding 350″ and the respective coding feature 351″ are incompatible with any of the counter coding 450 or counter coding feature 451 of the first type and are further incompatible with any of the counter coding 450′ or counter coding feature 451′ of the second type.

As further illustrated in FIGS. 25-28 the sidewall 202 of the receptacle 210 comprises a through recess 458 intersecting the coded groove 454 in radial direction. The insert 110 is provided with a visual indicator 358 matching in shape and size with the through recess 458. The indicator 358 is positioned on the outside surface 105 of the insert 110 such that when reaching a correct final assembly configuration, the indicator 358 overlaps with the through recess 458. Accordingly, and when correctly assembled the visual indicator 358 is visible through the through recess 458 from outside the housing 10. Typically, the visual indicator 358 distinguishes from the visual appearance of the outside surface 105 of the insert 110 at least by its color, brightness and/or texture.

In a further example of FIGS. 29 and 30 the first housing component 100 and the second housing component 200 are interconnectable by respective first and second connecting ends 101, 201. Here, the first housing component 100 is provided with an indicator 108 on the outside surface of the sidewall 102. The second housing components 200 is provided with a complementary shaped indicator 208. The indicators 108, 208 provide a visual guiding and control feature for the user of the drug delivery device 1. In this way and when reaching a final assembly configuration, the indicators 108, 208 align in longitudinal direction, thereby providing a visual feedback to the user, that the first and the second housing components 100, 200 are correctly assembled.

In principle, the fastening mechanism of the first housing component 100 and of the second housing component 200 is quite similar to the fastening mechanism as described above, e.g. in connection with any one of the FIGS. 3-28. The insert 110 is provided with a mechanical coding 550 comprising a coding feature 551 featuring a radial protrusion 552 protruding radially outwardly from the sidewall 102 of the insert 110. There is further provided a fastening element 520 implemented as a snap element 521 and featuring a radially outwardly extending projection 522 on the outside surface 105 of the insert 110. There are further provided first and second visual indicators 558, 559 that are provided at a circumferential offset from the fastening element 520, respectively.

As illustrated the fastening element 520 is located longitudinally adjacent the radially outwardly extending flange section 115. Hence, the fastening element 520 as well as the visual indicators 558, 559 are provided at a distal end of the insert 110.

There is further provided a radial protrusion 130 on the outside surface 105 of the insert 110. The protrusion 130 is longitudinally aligned with the fastening element 520. It is located longitudinally offset from the fastening element 520. It can be arranged at the same longitudinal position as the mechanical coding 550 but it is located circumferentially offset from the mechanical coding 550.

The receptacle 210 of the second housing component 200 is separately illustrated in FIG. 30. The receptacle 210 comprises an inside surface 203 of the sidewall 202 featuring a groove 624 and a groove 654. Both grooves 624, 654 comprises a first longitudinally extending groove portion 625, 655 and a second groove portion 626, 656. The groove 654 is a coding groove or coded groove and is configured to operate or to engage with the coding feature 551. The groove 654 is part of a radial recess 652 of a counter coding feature 651 of the mechanical counter coding 650 is provided on the inside surface 203 of the receptacle 210.

The groove 624 is configured to engage with the protrusion 130 and with the fastening element 520. The circumferential extent of the second groove portion 656 and of the second groove portion 626 is somewhat equivalent if not identical. This allows to perform the above-mentioned two-step assembly process. In a first step, the insert 110 is longitudinally inserted into the receptacle 210 without any rotation relative to the second housing component 200. Here, the protrusion 552 slides along the first groove portion 655 and the protrusion 130 as well as the projection 522 slide along the first groove portion 625. This insert motion in longitudinal direction may be blocked either when the flange section 115 abuts with the distal end face 214 of the receptacle 210 or when any one of the protrusions 130, 552 axially abuts with a longitudinal end of the first groove portion 625, 655.

Thereafter, the insert 110 can be subject to a rotation with regard to an axis of rotation extending parallel to an axis of symmetry of the first housing component 100 or second housing component 200. When in the intermediate assembly configuration, the visual indicator 558 can spatially overlap with a through recess 658 extending through the sidewall 202 of the receptacle 210 thereby indicating that the preassembly configuration has been reached.

Starting from the intermediate assembly configuration and by twisting the first housing component 100 relative to the second housing components 200 the protrusion 552 of the coding feature 551 slides along the second groove portion 656. The same applies to the protrusion 130. The protrusion 130 will slide along the circumferential extent of the second groove portion 626. The snap element 521 is located longitudinally offset from the second groove portion 626. When reaching the intermediate assembly configuration, the snap element 521 and its radially outwardly extending projection 522 is still located in the rather narrow portion of the first groove portion 625. Here, the side edge of the first groove portion 625 comprises a raised ridge 623, which exhibits a beveled side edge. Circumferentially offset from the ridge 623 there is provided a radial recess 622 which is sized and configured to positively engage with the projection 522 of the fastening element 520.

Contrary to the numerous examples as described above in connection with FIGS. 3-28 the counter fastening element 620 of FIG. 30 is not located inside the groove 424 but it is located circumferentially adjacent to the groove 624. The counter fastening element 620, which comprises a counter snap element 621, is configured to engage with the snap element 521. It is located at a circumferential offset from the first groove portion 625 and at a longitudinal offset from the second groove portion 626.

This implementation provides the benefit, that the fastening element 520 and the complementary-shaped counter fastening element 620 can be longitudinally shifted in close vicinity to the flange section 115. Accordingly, the counter fastening element 620 can be arranged near the distal end face 214 of the receptacle 210, which can facilitate a releasable or detachable engagement of the first and second housing components 100, 200.

When reaching a final assembly configuration, the further visual indicator 559 will align and overlap with the through recess 658, thereby indicating, that a final assembly configuration has been reached. Since the second visual indicator 559 is visible through the through recess 658 from outside the housing 10 a respective visual feedback can be provided to the user. Typically, the visual indicator 558 and the visual indicator 559 mutually distinguish by at least one of a color, a brightness and/or texture. They are visually distinguishable.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the mechanical coding, the mechanical counter coding or as described in connection with the protrusion and the groove or in connection with the fastening element and counter fastening element can be interchanged provided and implemented in an inverted way compared to the presently shown examples.

The receptacle 210 as shown in FIG. 30 can be integrated into the tubular sidewall 202 of the second housing component 200. Alternatively, it can be provided by a housing insert 206 fixedly attachable or fixedly attached to housing component 200. The housing insert 206 can be rotationally and/or longitudinally fixed to the elongated housing component 200. Insofar all features and benefits as described above in connection with the receptacle equally apply to a housing insert fixedly connectable or fixedly connected to a respective housing component.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the mechanical coding, the mechanical counter coding or as described in connection with the protrusion and the groove or in connection with the fastening element and counter fastening element can be interchanged provided and implemented in an inverted way compared to the presently shown examples.

### REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
105 outside surface
108 indicator
110 insert
112 end face
114 abutment face
115 flange section
120 fastening element
121 snap element
122 protrusion
124 tongue
125 chamfer
126 slit
130 protrusion
150 mechanical coding
151 coding feature
152 radial protrusion
200 housing component
201 connecting end
202 sidewall
203 inside surface
205 outside surface
206 housing insert
210 receptacle
211 insert opening
212 end face
214 end face
215 sidewall portion
220 counter fastening element
221 counter snap element
222 recess
230 groove
250 mechanical counter coding
251 counter coding feature
252 radial recess
320 fastening element
321 snap element
322 projection
350 mechanical coding
351 coding feature
352 radial protrusion
358 indicator
420 counter fastening element
421 counter snap element
422 recess
423 ridge
424 groove
425 groove portion
426 groove portion
450 mechanical counter coding
451 counter coding feature
452 radial recess
454 coded groove
455 groove portion
456 groove portion
458 through recess
550 mechanical coding
551 coding feature
552 radial protrusion
558 indicator
559 indicator
620 counter fastening element
621 counter snap element
622 recess
623 ridge
624 groove
625 groove portion
626 groove portion
650 mechanical counter coding
651 counter coding feature
652 radial recess
654 groove
655 groove portion
656 groove portion
658 through recess

1.-15. (canceled)

16. A housing of a drug delivery device, the housing comprising:
a first housing component configured to accommodate a cartridge filled with a medicament and comprising a first connecting end,
a second housing component configured to accommodate a drive mechanism of the drug delivery device and comprising a second connecting end,
an insert provided on one of the first connecting end or the second connecting end,
a receptacle provided on the other one of the first connecting end or the second connecting end, wherein the insert is insertable into the receptacle along a longitudinal direction for mutually fastening the first housing component and the second housing component,
a fastening element provided on the insert,
a counter fastening element complementary shaped to the fastening element and provided in the receptacle,
a mechanical coding provided on the insert and comprising a coding feature,
a mechanical counter coding provided in the receptacle and comprising a counter coding feature,
the mechanical coding and the mechanical counter coding being operable to prevent an engagement of the fastening element with the counter fastening element when the mechanical coding does not match the mechanical counter coding,
wherein the mechanical coding is defined by a combination of a position of the coding feature with regard to the longitudinal direction and an extent of the coding feature in at least one of the longitudinal direction or a transverse direction.

17. The housing according to claim 16, wherein the coding feature and the counter coding feature comprise:
a radial protrusion on one of the insert or the receptacle; and
a radial recess on the other one of the insert or the receptacle.

18. The housing according to claim 17, wherein when the mechanical coding matches the mechanical counter coding, the radial protrusion fits into the radial recess.

19. The housing according to claim 17, wherein when the mechanical coding does not match the mechanical counter coding, the radial protrusion does not fit into the radial recess.

20. The housing according to claim 17, wherein the coding feature is a longitudinal position versus a longitudinal extent of one of the radial protrusion or the radial recess on an outside surface of the insert.

21. The housing according to claim 17, wherein the coding feature is a longitudinal position versus a transverse extent of one of the radial protrusion or the radial recess on an outside surface of the insert.

22. The housing according to claim 17, wherein the coding feature and the counter coding feature distinguish from a second coding feature and a second counter coding feature of a second housing by varying the longitudinal position of the radial recess and the longitudinal position of the radial protrusion correspondingly, wherein an increase of a longitudinal distance of the radial recess from a free end of one of the first connecting end or the second connecting end is accompanied by an increase in at least one of the longitudinal extent or the transverse extent of the radial recess and by a corresponding decrease of a distance of the radial protrusion from the other one of the first connecting end or the second connecting end and a corresponding increase of the longitudinal extent or the transverse extent of the radial protrusion.

23. The housing according to claim 17, further comprising:
a groove provided on one of the insert or the receptacle, the groove extending along the longitudinal direction, and
a protrusion provided on the other one of the insert or the receptacle, the receptacle configured to slide along the groove upon insertion of the insert into the receptacle to rotationally lock the first housing component relative to the second housing component.

24. The housing according to claim 17, wherein the radial recess comprises a coded groove, and wherein:
the coded groove is provided on an inside surface of a sidewall of the receptacle and adjoins a longitudinal end face of the second connecting end, or
the coded groove is provided on an outside surface of a sidewall of the insert and adjoins a longitudinal end face of the first connecting end.

25. The housing according to claim 24, wherein the coded groove comprises a first groove portion and a second groove portion, wherein the first groove portion extends along the longitudinal direction and adjoins the longitudinal end face of one of the first connecting end or the second connecting end, and wherein the second groove portion extends along a circumferential direction and merges into the first groove portion at a longitudinal distance from the longitudinal end face of one of the first connecting end or the second connecting end.

26. The housing according to claim 25, wherein the coding feature and the counter coding feature distinguish from a second coding feature and a second counter coding feature of a second housing by varying the longitudinal position of the second groove portion and the longitudinal position of the radial protrusion correspondingly, wherein an increase of a longitudinal distance of the second groove portion from the longitudinal end face of one of the first connecting end or the second connecting end is accompanied by an increase of the longitudinal width of the second groove portion and by a corresponding increase of the longitudinal extent of the corresponding radial protrusion.

27. The housing according to claim 16, wherein the fastening element comprises a snap element and the counter fastening element comprises a counter snap element having a complementary shape to the snap element, and wherein the snap element is configured to engage with the counter snap element to interlock the first and second housing components against a relative movement in the longitudinal direction or the circumferential direction.

28. The housing according to claim 27, wherein the mechanical coding is integrated into the snap element and is defined by a longitudinal position and one of a longitudinal extent or a transverse extent of the snap element on the insert.

29. The housing according to claim 27, wherein the mechanical counter coding is integrated into the counter snap element and is defined by a longitudinal position and one of a longitudinal extent or a transverse extent of the counter snap element in the receptacle.

30. The housing according to claim 16, further comprising:
a groove provided on one of the insert or the receptacle, the groove extending along the longitudinal direction, and
a protrusion provided on the other one of the insert or the receptacle, the receptacle configured to slide along the groove upon insertion of the insert into the receptacle to rotationally lock the first housing component relative to the second housing component.

31. An injection device for injecting a dose of a medicament, the injection device comprising:
a housing comprising:
a first housing component configured to accommodate a cartridge filled with a medicament and comprising a first connecting end,
a second housing component configured to accommodate a drive mechanism of the drug delivery device and comprising a second connecting end,
an insert provided on one of the first connecting end or the second connecting end,
a receptacle provided on the other one of the first connecting end or the second connecting end, wherein the insert is insertable into the receptacle along a longitudinal direction for mutually fastening the first housing component and the second housing component,
a fastening element provided on the insert,
a counter fastening element complementary shaped to the fastening element and provided in the receptacle,
a mechanical coding provided on the insert and comprising a coding feature,
a mechanical counter coding provided in the receptacle and comprising a counter coding feature,
the mechanical coding and the mechanical counter coding being operable to prevent an engagement of the fastening element with the counter fastening element when the mechanical coding does not match the mechanical counter coding,
wherein the mechanical coding is defined by a combination of a position of the coding feature with regard to the longitudinal direction and an extent of the coding feature in at least one of the longitudinal direction or a transverse direction; and
the cartridge arranged inside the housing, the cartridge comprising a barrel filled with the medicament and sealed in a proximal longitudinal direction by a movable bung, and
the drive mechanism arranged inside the housing, the drive mechanism comprising a piston rod operable to exert a distally directed dispensing force onto the bung of the cartridge.

32. A kit comprising:
a first housing comprising:
a first housing component configured to accommodate a cartridge filled with a medicament and comprising a first connecting end,
a second housing component configured to accommodate a drive mechanism of a drug delivery device and comprising a second connecting end,
a first insert provided on one of the first connecting end or the second connecting end,
a first receptacle provided on the other one of the first connecting end or the second connecting end, wherein the first insert is insertable into the first receptacle along a longitudinal direction for mutually fastening the first housing component and the second housing component,
a first fastening element provided on the first insert,
a first counter fastening element complementary shaped to the first fastening element and provided in the first receptacle,
a first mechanical coding provided on the first insert and comprising a first coding feature,
a first mechanical counter coding provided in the first receptacle and comprising a first counter coding feature,
the first mechanical coding and the first mechanical counter coding being operable to prevent an engagement of the first fastening element with the first counter fastening element when the first mechanical coding does not match the first mechanical counter coding,
wherein the first mechanical coding is defined by a combination of a position of the first coding feature with regard to the longitudinal direction and an extent of the first coding feature in at least one of the longitudinal direction or a transverse direction; and
a second housing comprising:
a third housing component configured to accommodate a second cartridge filled with a second medicament and comprising a third connecting end,
a fourth housing component configured to accommodate a second drive mechanism of the drug delivery device and comprising a fourth connecting end,
a second insert provided on one of the third connecting end or the fourth connecting end,
a second receptacle provided on the other one of the third connecting end or the fourth connecting end, wherein the second insert is insertable into the second receptacle along a second longitudinal direction for mutually fastening the third housing component and the fourth housing component,
a second fastening element provided on the second insert,
a second counter fastening element complementary shaped to the second fastening element and provided in the second receptacle,
a second mechanical coding provided on the second insert and comprising a second coding feature,
a second mechanical counter coding provided in the second receptacle and comprising a second counter coding feature,
the second mechanical coding and the second mechanical counter coding being operable to prevent an engagement of the second fastening element with the second counter fastening element when the second mechanical coding does not match the second mechanical counter coding,
wherein the second mechanical coding is defined by a combination of a second position of the second coding feature with regard to the second longitudinal direction and a second extent of the second coding feature in at least one of the second longitudinal direction or a second transverse direction;
wherein the first coding feature of the first housing distinguishes from the second coding feature of the second housing by its position with regard to the longitudinal direction and by its extent in at least one of the longitudinal direction or transverse direction.
